(19) United States
(12) Patent Application Publication
(10) Pub. No.: US 2026/0325358 A1
(43) Pub. Date: Oct. 22, 2026

(54) Dissolvable Antimicrobial Medical Patch (DAMP) for Emergency Wound Treatment

(71) Applicants: Vikas Ravjibhai Patel, Morrisville, NC (US); Chandra Obula Reddy Kanala, Cary, NC (US); Aarav Vakani, Cary, NC (US)

(72) Inventors: Shiv Vikas Patel, Morrisville, NC (US); Jagadeeswar Reddy Kanala, Cary, NC (US); Aarav Vakani, Cary, NC (US)

(21) Appl. No.: 19/316,333

(22) Filed: Sep. 2, 2025

(51) Int. Cl. A61F 13/0203; A61F 13/00; A61F 13/51

(52) U.S. Cl. CPC A61F 13/0213; A61F 13/51; A61F 13/00063; A61F 2013/00106; A61F 2013/00157; A61F 2013/00748; A61F 2013/00927; A61F 2013/51035

(57) ABSTRACT

A fully dissolvable wound-care patch is disclosed, comprising multiple functional layers that dissolve sequentially to provide controlled wound treatment without removal or re-application. The patch includes a hydrocolloid adhesive layer of pectin, carboxymethylcellulose, and gelatin to secure the patch and maintain a moist environment; a honey layer providing antibacterial and anti-inflammatory effects; a sodium alginate layer forming a gel and absorbing exudate; a chitosan layer delivering antimicrobial and regenerative support; and a collagen layer acting as a scaffold to promote skin regeneration and reduce scarring. A gelatin-polyvinyl alcohol film regulates dissolution between therapeutic layers, and a medical-grade gauze layer shields the wound externally. The patch is biodegradable, leaving only gauze after therapy, and is suitable for emergency and remote-care applications.

### FIELD OF THE INVENTION

The present invention pertains to biodegradable, dissolvable multi-layer antimicrobial wound patches for remote, emergency, or resource-limited environments where changing dressings is impractical.

### BACKGROUND OF THE INVENTION

Existing multi-layer wound dressings typically maintain structural integrity or conformability, but do not dissolve sequentially and are not designed for self-contained, staged release of therapeutic agents without intervention.

### SUMMARY OF THE INVENTION

The invention relates to a fully dissolvable wound-care patch that provides staged therapeutic action through sequentially dissolving layers, thereby eliminating the need for removal or re-application during treatment. The patch is designed for wound environments where moisture management, infection control, and tissue regeneration must be achieved in a controlled, time-dependent manner.

The patch comprises a hydrocolloid adhesive base layer composed of pectin, carboxymethylcellulose, and gelatin, which secures the device to the skin and maintains a moist environment conducive to healing. Above the adhesive is a honey layer that imparts antibacterial and anti-inflammatory effects to reduce infection risk and swelling. A sodium alginate layer follows, forming a gel matrix that absorbs exudate and retains moisture. A chitosan layer provides antimicrobial protection and supports tissue regeneration, while a collagen layer serves as a biological scaffold to enhance cellular repair and minimize scar formation.

A gelatin-polyvinyl alcohol (PVA) film layer is positioned between the therapeutic layers to regulate dissolution and maintain separation of active components, thereby ensuring orderly timed release. The outermost surface of the patch includes a medical-grade gauze layer that protects the wound from external contaminants while allowing controlled exposure of the underlying layers to wound moisture.

The therapeutic layers are configured to dissolve sequentially in response to wound conditions, delivering extended treatment over a period of hours to days. Upon completion of therapy, the biodegradable layers fully dissolve, leaving only the gauze component. The invention is particularly suited for use in emergency medicine, disaster relief, and remote environments where conventional wound dressings may not be practical.

### DETAILED DESCRIPTION OF THE INVENTION

The patch consists of sequential layers extending from the wound-contact surface outward.

A hydrocolloid adhesive layer, composed of pectin, carboxymethylcellulose, and gelatin, ensures adhesion to the skin and maintains a moist wound environment.

Above the adhesive, a honey layer provides natural antibacterial and anti-inflammatory activity.

A sodium alginate layer absorbs wound exudate and forms a gel barrier to retain moisture.

A chitosan layer contributes antimicrobial properties and supports cellular regeneration.

A collagen layer acts as a biological scaffold to facilitate skin cell attachment, encourage tissue repair, and minimize scarring.

A gelatin-polyvinyl alcohol (PVA) film layer is interposed between therapeutic layers to regulate dissolution timing and prevent premature mixing of active components.

The outermost surface comprises a medical-grade gauze layer, which protects the wound from environmental contaminants while allowing sequential exposure of underlying layers to wound moisture.

In one embodiment, the patch is square with approximate dimensions of 50 mm×50 mm. Other embodiments include alternative geometries such as rectangular or circular shapes, with sizes ranging from 25 mm×25 mm to 100 mm×100 mm.

### BRIEF DESCRIPTION OF THE DRAWINGS

FIG. 1 is an angled view of top and side of the dissolvable antimicrobial wound patch showing an exemplary square configuration of approximately 50 mm×50 mm and total thickness of 6.2 mm.

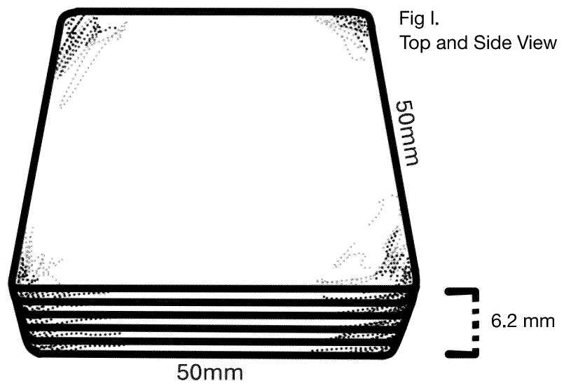

FIG. 2 is a top view of the dissolvable antimicrobial wound patch showing an exemplary square configuration of approximately 50 mm×50 mm.

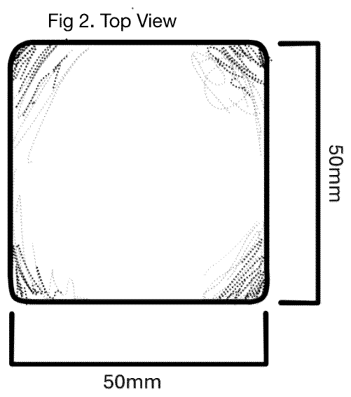

FIG. 3 is an exploded schematic view of the dissolvable antimicrobial wound patch showing each functional layer in sequential order including thickness of each layer.

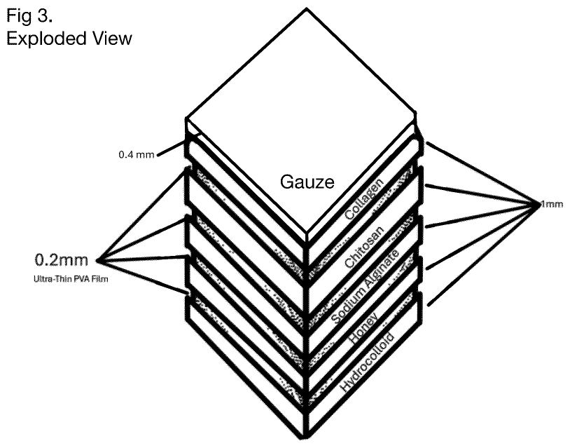

FIG. 4 is a schematic side view of the dissolvable antimicrobial wound patch showing thickness measurement of each layer.

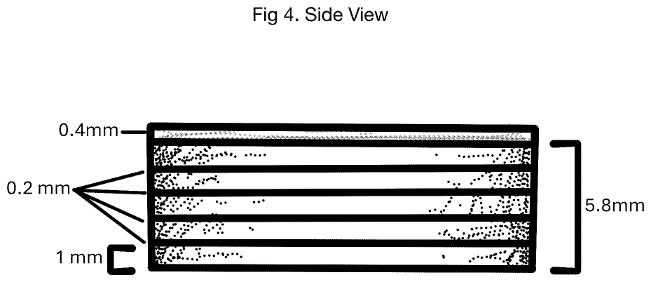

What is claimed is:

1. A biodegradable dissolvable multi-layer antimicrobial wound patch that leaves gauze on the skin, comprising, in sequential order, (a) a hydrocolloid adhesive layer comprising pectin, carboxymethylcellulose, and gelatin, maintaining skin adhesion and a moist environment at the very bottom; (b) a honey layer providing antibacterial and anti-inflammatory effects; (c) a sodium alginate layer forming a moisture-retentive gel and absorbing exudate; (d) a chitosan layer providing antimicrobial and regenerative support; (e) a collagen layer serving as a scaffold to support skin regeneration and reduce scarring; (f) a gelatin-polyvinyl alcohol film layer dissolving to control timed release, wherein this layer sits between all the medical layers and acts as a barrier; and (g) a medical-grade gauze layer that protects the healing skin from outside influence; wherein each layer dissolves sequentially when in contact with wound moisture without requiring dressing changes.

2. The patch of claim 1, wherein each layer dissolves at a predetermined time interval, the hydrocolloid adhesive dissolving in about 12 hours, the honey layer in about 5 to 6 hours, the sodium alginate layer in about 3 to 4 hours, the chitosan layer in about 1 to 2 hours, the collagen layer in about 8 to 10 hours, and the gelatin-polyvinyl alcohol film layer in about 15 to 30 minutes, thereby enabling a total therapeutic release duration of up to 36 hours.

3. The patch of claim 1, wherein layer thicknesses are about 1.0 mm for the hydrocolloid adhesive, about 1.0 mm for the honey, about 1.0 mm for the sodium alginate, about 1.0 mm for the chitosan, about 1.0 mm for the collagen, and about 0.2 mm for each gelatin-polyvinyl alcohol film layer.

4. The patch of claim 1, wherein the patch is square and has dimensions of approximately 50 mm by 50 mm.

5. The patch of claim 1, wherein alternative shapes include circular and rectangular, in sizes ranging from approximately 25 mm by 25 mm to 100 mm by 100 mm.

6. The patch of claim 1, wherein the patch is fully biodegradable and dissolves entirely upon wound moisture contact, excluding the gauze layer.

7. The patch of claim 1, wherein the hydrocolloid adhesive comprises pectin, carboxymethylcellulose, and gelatin in effective ratios.

8. The patch of claim 1, wherein the patch eliminates dressing changes for up to 36 hours post-application.

9. A method for treating a wound, comprising cleaning the wound site, dampening the wound with water, applying the patch of claim 1, and allowing the layers to dissolve sequentially, thereby releasing antimicrobial, healing, and moisture-retaining agents without further intervention.

10. The method of claim 9, wherein the wound is a first-degree burn or a second-degree burn and the patch maintains a moist antimicrobial environment to accelerate tissue regeneration and reduce scarring.
